(19) United States
(12) Patent Application Publication
(10) Pub. No.: US 2026/0176646 A1
(43) Pub. Date: Jun. 25, 2026

(54) TRANSGENIC PLANTS AND METHODS OF MAKING AND USING

(71) Applicant: NUtech Ventures, Lincoln, NE (US)

(72) Inventors: Panya Kim, Lincoln, NE (US); Hanh T. Nguyen, Lincoln, NE (US); Matthew Sousek, Lincoln, NE (US); Roch E. Gaussion, Lincoln, NE (US); James R. Alfano, Lincoln, NE (US); Ming Guo, Lincoln, NE (US); Thomas E. Clemente, Lincoln, NE (US)

(21) Appl. No.: 19/196,420

(22) Filed: May 1, 2025

Related U.S. Application Data
(60) Provisional application No. 63/641,357, filed on May 1, 2024.

(51) Int. Cl. C12N 15/82

(52) U.S. Cl. CPC C12N 15/8261; C12N 15/8282

(57) ABSTRACT

This disclosure describes transgenic plants overexpressing a microtubule-associated protein 65-1 (MAP65-1), as well as methods of making and using such plants.

### INCORPORATION BY REFERENCE

This application contains a Sequence Listing that has been submitted electronically as an XML file named “24742-0150001_SL_ST26.xml.” The XML file, created on Jul. 14, 2025, is 9.01 bytes in size. The material in the XML file is hereby incorporated by reference in its entirety.

### CROSS-REFERENCE TO RELATED APPLICATIONS

This application claims the benefit of priority under 35 U.S.C. § 119 (e) to U.S. Application No. 63/641,357 filed on May 1, 2024.

### FEDERALLY SPONSORED RESEARCH OR DEVELOPMENT

This invention was made with government support under 1758459 awarded by the National Science Foundation. The government has certain rights in the invention.

### TECHNICAL FIELD

This disclosure generally relates to transgenic plants and methods of making and using such plants.

### BACKGROUND

MAP65-1 is a microtubule-associated protein (MAP) that is involved in regulating plant cell growth and responding to stress. MAP65-1 contributes to axial cell growth in the expanding hypocotyl and is involved in the rapid depolymerization and reorganization of cortical microtubules in response to salt stress. Additionally, MAP65-1 has been shown to be involved in plant immunity.

### SUMMARY

This document reports on the development and characterization of transgenic soybean plants that overexpress MAP65-1 protein homologs from Arabidopsis or soybean and the phenotypic outcomes associated with biotic and abiotic challenges to selected events.

Specifically, transgenic soybean seeds were generated in which the expression of a microtubule-associated protein, MAP65-1, is enhanced. Transgenic alleles that encode for the Arabidopsis MAP65-1 (AtMAP65-1) along with the soybean MAP65-1 (GmMAP65-1), corresponding to gene models At5g55230 and Glyma.02g295100, for the Arabidopsis and soybean homologs, respectively, were assembled into constitutive expression cassettes and introduced into soybean (Glycine max Merr L.). As described herein, transgenic plants displayed a resistance phenotype to an oomycete pathogen, Phytophthora sojae, to bacterial pathogens, Pseudomonas syringae pv glycinea strains, and increased tolerance to the herbicide, oryzalin.

In one aspect, constructs are provided that include a nucleic acid sequence encoding a MAP65-1 protein under direction of a promoter. In some embodiments, the nucleic acid encoding the MAP65-1 protein includes the sequence shown in SEQ ID NO:1 or 3 or a sequence having at least 90% sequence identity to SEQ ID NO:1 or 3. In some embodiments, the MAP65-1 protein includes the sequence shown in SEQ ID NO:2 or 4 or a sequence having at least 90% sequence identity to SEQ ID NO:2 or 4.

In some embodiments, the promoter is a constitutive promoter. In some embodiments, the construct further includes one or more regulatory elements.

In some embodiments, a cell is provided that includes a construct as described herein. In some embodiments, the cell is a plant cell. In some embodiments, the cell is a bacterial cell.

In another aspect, transgenic plants are provided that include an exogenous nucleic acid encoding a microtubule-associated protein 65-1 (MAP65-1) protein. In some embodiments, the exogenous nucleic acid encoding the MAP65-1 protein includes the sequence shown in SEQ ID NO: 1 or 3 or a sequence having at least 90% sequence identity to SEQ ID NO:1 or 3.

In some embodiments, the transgenic plant exhibits increased resistance to Pseudomonas syringae pv. Glycinea relative to a corresponding plant lacking the exogenous nucleic acid. In some embodiments, the transgenic plant exhibits increased resistance to Phytophthora sojae relative to a corresponding plant lacking the exogenous nucleic acid. In some embodiments, the transgenic plant exhibits increased tolerance to herbicides that destabilize microtubules relative to a corresponding plant lacking the exogenous nucleic acid.

In some embodiments, the exogenous nucleic acid further includes a promoter. In some embodiments, the promoter is a constitutive promoter.

In some embodiments, the transgenic plant is a soybean plant.

In yet another aspect, methods of making a transgenic plant are provided. Such methods typically include introducing a construct as described herein into a plant cell to generate a transgenic plant cell; and regenerating the transgenic plant cell into a transgenic plant.

In some embodiments, the transgenic plant exhibits increased resistance to Pseudomonas syringae pv. Glycinea relative to a plant lacking the exogenous nucleic acid encoding the MAP65-1 protein. In some embodiments, the transgenic plant exhibits increased resistance to Phytophthora sojae relative to a plant lacking the exogenous nucleic acid encoding the MAP65-1 protein. In some embodiments, the transgenic plant exhibits increased tolerance to herbicides that destabilize microtubules relative to a plant lacking the exogenous nucleic acid encoding the MAP65-1 protein.

In some embodiments, the transgenic plant is a soybean plant.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a phylogenic tree of MAP65 proteins from Arabidopsis and soybean. A maximum likelihood tree was built using MEGA software with 2000 bootstraps. CRE14. g614050 is a MAP65-1-like protein from Chlamydomonas reinhardtii and is included as an outgroup. The red boxes indicate the two MAP65-1 alleles that were introduced in soybean transgenic plants.

FIG. 2 are graphs showing that soybean MAP65-1 transgenic plants are more resistant to P. syringae pv. glycinea LN10. Soybean plants (2A), or oryzalin-treated soybean plants (2B), were vacuum-infiltrated with a bacterial suspension of P. syringae pv. glycinea LN10 (1×10⁵ cell/ml) with 0.02% Silwet®. Wild type soybean genotype Thorne, AtMAP65-1 (event 1107 Jan. 1) and GmMAP65-1 (event 1130 Jan. 26) soybean transgenic plants were used for the bacterial growth assays. Bacterial growth in leaves was determined at 0 and 3 day post inoculation (dpi). Bacterial growth means are represented (n=4) at days 0 and 3. Error bars indicate SD. Statistical analysis was performed with ANOVA followed by LSD mean separation with different letters indicating statistical significance (P<0.05).

FIG. 3 shows the results of a pathogenicity assay of Phytophthora sojae on MAP65-1 soybean transgenic plants. (3A) images showing the symptom of P. sojae on soybean hypocotyls infected with P. sojae. The elongated hypocotyls grown in dark were wound-inoculated by microinjection with 100 zoospores of the indicated P. sojae strains. The bent hypocotyl symptoms were recorded 2 days after inoculation. Wild type Thorne, AtMAP65-1 (event 1107 Jan. 1) and GmMAP65-1 (event 1130 Jan. 26) overexpressing soybean transgenic events were used in the pathogenicity assays. (3B) a graph showing the abundance of PsACT1 of P. sojae strains determined with quantitative ddPCR that reflects relative biomass of P. sojae in soybean plants infected with Ps6497 and PsNE-21 strains. Values are means±SEM (n=3). Asterisks indicate statistical significance (p<0.05). Statistical differences were performed student t-test (P<0.05).

FIG. 4 is data showing oryzalin tolerance assay in soybean plants. (4A) images showing results of assays with 10-day-old soybean seedling grown on MS medium supplemented with oryzalin at indicated concentrations. (4B) images showing four-week-old soybean plants applied with oryzalin of indicated amount calculated based on the commercial herbicide Surflan® that contains 40.4% oryzalin. (4C-4D) graphs showing leaf damages quantified for treated plants at seedling stage (4C) and emergency stage (4D). The concentration of 1× Surflan® is equivalent to 0.532 g/l of active reagent oryzalin. Wild type Thorne, AtMAP65-1 (event 1107 Jan. 1) and GmMAP65-1 (event 1130 Jan. 26) soybean transgenic plants were used in both assays. Values are means (n=4)+SD. Statistical analysis was performed with ANOVA followed by LSD mean separation (P<0.05).

FIG. 5 depicts results showing that AtMAP65-1 and GmMAP65-1 soybean plants maintain root biomass in low temperature stress. (5A) a graph showing the weight of root parts with different temperature treatments; (5B) a representative image of plants for measurements in (5A). Wild type and soybean plants overexpressing AtMAP65-1 (events 1107 Jan. 1 and 1107 Feb. 2) and GmMAP65-1 (events 1130 Jan. 26 and 1134 Jan. 7) were either grown treated at normal growing temperature (24° C.) or treated at low temperature (4° C.) for 10 days. Values are means (n=4)+SD. Statistical analysis was performed with ANOVA followed by LSD mean separation (P<0.05).

FIG. 6 is a graph showing that GmMAP65-1 soybean plants are slightly resistant to freezing stress. Ion leakage assays with wild type Thorne, transgenic plants overexpressing AtMAP65-1 (event 1107 Jan. 1), and GmMAP65-1 (event 1130 Jan. 26). Freezing tolerance was quantified as ion leakage in three-week-old soybean plants that was calculated as the percentage relative to the total ion leakage.

FIG. 7 demonstrates the confirmation of MAP65-1 soybean transgenic plants. (7A) is an image of three-week-old soybean plant leaves that were painted with a Q-tip soaked with Basta solution at a concentration of 200 mg/L. A representative picture taken 2 days after painting shows Thorne is sensitive and a soybean transgenic event is resistant in the presence of Basta gene. (7B) shows immunoblots of soybean transgenic plants. Anti-HA antibody was used to detect MAP65-1 proteins in AtMAP65-1 (events 1107 Jan. 1, 1107 Jan. 20, 1107 Feb. 2, and 1107 Feb. 4) and GmMAP65-1 (events a1130-1-23, 1130 Jan. 26, and 1134 Jan. 7) transgenic soybean events.

FIG. 8 are graphs showing the results of soybean cyst nematode (SCN) bioassays. Both SCN reproductive factors (8A) and plant height of infected plants (8B) are not affected. AtMAP65-1 (Line 1107 Jan. 1) and GmMAP65-1 (Line 1130 Jan. 26) transgenic soybean plants, wild type Thorne, and Williams 82 (a susceptible control) were inoculated with SCN. No significant difference was seen in the assay.

FIG. 9 are graphs showing plant biomass in cold stress. (9A) shows the weight of total plants, (9B) shows the weight of aerial parts with different temperature treatments. Values are means+SD (n=4). Individual plants at 24° C. and at 4° C. Statistical analysis was conducted with ANOVA followed by LSD mean separation (P<0.05). Wild type and soybean plants overexpressing AtMAP65-1 (Line1107-1-1 and 1107 Feb. 2) and GmMAP65-1 (Line1130-1-26 and 1134 Jan. 7) were grown either at normal growing temperature (24° C.) or at cold temperature (4° C.) for 10 days. The experiment was repeated twice with similar results.

FIG. 10 is an alignment of MAP65's C-terminal microtubule binding motifs. Bold letters showed conserved amino acids. Asterisks showed critical residues for microtubule binding. Asterisks indicate the conserved alanine residues required for microtubule binding. (SEQ ID NOs: 5-24 (top to bottom)).

### DETAILED DESCRIPTION

Microtubule-associated protein 65-1 (MAP65-1) protein plays an essential role in plant cellular dynamics through impacting stabilization of the cytoskeleton by serving as a crosslinker of microtubules. The role of MAP65-1 in plants has been associated with phenotypic outcomes in response to various environmental stresses. The Arabidopsis MAP65-1 (AtMAP65-1) is a known virulence target of plant bacterial pathogens and, thus, a component of plant immunity. Soybean events were generated that carry transgenic alleles for both AtMAP65-1 and GmMAP65-1, the soybean AtMAP65-1 homolog, under control of cauliflower mosaic virus 35S promoter. Both AtMAP65-1 and GmMAP65-1 transgenic soybeans were more resistant than wild type plants to challenges by the soybean bacterial pathogen Pseudomonas syringae pv. glycinea and the oomycete pathogen Phytophthora sojae, but not the soybean cyst nematode, Heterodera glycines. Soybean plants expressing AtMAP65-1 and GmMAP65-1 also display a tolerance to the herbicide, oryzalin, which has a mode of action that destabilizes microtubules. In addition, GmMAP65-1 expressing soybean plants show reduced cytosol ion leakage under freezing conditions, suggesting that ectopic expression of GmMAP65-1 may enhance cold tolerance in soybean. Taken together, over-expression of AtMAP65-1 or GmMAP65-1 confers tolerance of soybean plants to varieties of biotic and abiotic stresses.

### Microtubule-Associated Proteins (MAPs)

Cytoskeleton microtubules play essential roles in stability of cellular integrity, division, and organellar movement. Microtubule-associated proteins (MAPs), categorized as motor and non-motor proteins, help maintain cell dynamics for proper growth by ensuring accurate arrangement of microtubules to implement specific molecular and cellular functions.

The non-motor Ase1/PRC1/MAP65 family proteins are broadly conserved and present in all eukaryotes. These include the microtubule associated protein 65 (MAP65) in plants, and the MAP65 homologs, midzone-specific protein anaphase spindle elongation (Ase1) in yeast, and protein regulating cytokinesis 1 (PRC1) in human. The commonality among these proteins is the ability to bind microtubules.

Multiple MAP65 homologs have been identified and appear to be conserved among flowering plant species. Nine MAP65 homologs (MAP65-1 to -9) share 28-79% sequence identity in Arabidopsis, while 11 OsMAP65 genes are present in the rice genome.

Like in yeast and animals, MAP65 proteins act preferentially as crosslinker of anti-parallel microtubules (MTs) to stabilize the cellular MTs matrix. MAP65 proteins bind MTs to manifest microtubule bundling through interaction with the carboxy-terminus of tubulin, that promotes microtubule assembly.

Although they commonly serve as microtubule crosslinkers, MAP65 protein members differ in several ways. They harbor divergent sequences at the C-termini and their localization patterns can vary.

MAP65 proteins act as a microtubule stabilizer that coordinates microtubule bundling and growth. NtMAP65-1 in tobacco and AtMAP65-2 in Arabidopsis both interact with microtubule bundles, stabilize them, and prevent the bundled microtubules from severing. MAP65 proteins maintain the integrity of both the midzone microtubules within the central spindle assembly, and the phragmoplast, which is essential for successful completion of, while some MAP65 proteins are also functionally redundant during cytokinesis.

MAP65 proteins are likely also involved in cell expansion after cell division, participating in the establishment of cortical microtubule arrays. Both AtMAP65-1 and AtMAP65-2 have been shown to promote axial cell growth in etiolated hypocotyls as well as roots of Arabidopsis, with functional redundancy.

Tissue and/or organ specificity of MAP65 proteins have been observed. An Arabidopsis AtMAP65-3/ple mutant allele displays an abnormal morphogenesis with short and irregular expanded root system that coincides with enlarged multinucleated cells, indicating complete karyokinesis but incomplete defective cytokinesis in an organ specific manner.

The maize ZmMAP65-3 is required for cellularization of the syncytial embryo sac, a function the AtMAP65-3 does not have in Arabidopsis, reflecting the variation in biological roles across plant species.

MAP65 proteins also have been shown to play a role in responses to challenges by various plant-associated microorganisms, including phytopathogenic and symbiotic microbes. AtMAP65-1 was demonstrated to be a virulence target in plants of the effector protein HopE1 from the phytobacterial pathogen Pseudomonas syringae pv. tomato DC3000 (P. syringae DC3000). HopE1 interacts with AtMAP65-1 in a calmodulin-dependent manner and mediates dissociation from microtubules, leading to impaired secretion of pathogenesis-related (PR) proteins into the apoplast, indicating that AtMAP65-1 may be a component of plant immunity against plant bacterial pathogens.

This is consistent with the maize ZmMAP65-la being a positive regulator of H₂O₂ amplification and enhancing brassinosteroid-induced antioxidant defense response in maize. By contrast, other MAP65 homologs may promote disease of different types of pathogens. For, example, during induction of root knot formation, MAP65-3 is essential for nematode-induced giant cell ontogenesis required for reproduction of the parasite but does not affect feeding establishment or symptom development in Arabidopsis. MAP65-3 has also been shown to support infection by filamentous biotrophic pathogens.

In the soybean symbiotic microbial relationship, two MAP65-1 homologs along with GmMAP3 and GmMAP4 are activated in young nodules displaying elevated expression in roots. In the legume Medicago truncatula, the MtMAP65-1 protein is phosphorylated by Aurora kinase 1 (MtAUR1), leading to the formation of AUR1-MAP65 mitotic module recruitment in root hairs infected with endosymbiotic bacteria Sinorhizobium meliloti, consistent with MtMAP65's role in symbiotic interaction and nodulation in legume plants.

Microtubule dynamics has also been associated with plant's adaptation to low temperature abiotic stress. Microtubule structure is critical to the low temperature response, and the cytoskeleton is remodeled during low temperatures, suggesting that microtubule dynamics through MAP65-1 may be necessary. Microtubule depolymerization can be induced by cold stress, and MAP65-1 alleles have been associated with tolerance to a cold stress challenge.

The herbicide oryzalin, 3,5-dinitro-N⁴,N⁴-dipropylsulfanilamide, binds plant tubulin, leading to inhibition of microtubule polymerization, triggering depolymerization of microtubule organization in plants. Whether oryzalin directly acts on MAP65 proteins is not known, given that the sensitivity to the microtubule depolymerizing induced by oryzalin was indiscriminative between wild type and Atmap65-1/-2 mutants of Arabidopsis. However, the filamentous image pattern of GFP-AtMAP65-1 can be disrupted by oryzalin. While transient expression of AtMAP65-3 maintains a more stable microtubule bundling, as does transient expression of AtMAP65-5, upon application of oryzalin, the transient activity of phospholipase D1 (PLD1) is increased. PLD1 produces phosphatidic acid that, in turn, binds MAP65-1, suggesting oryzalin may have an indirect effect on MAP65 protein. Despite its herbicidal activity, oryzalin-mediated disruption of microtubules could result in an increase of the survival rate of seedlings under salt stress, which may be related to its ability to release MAP65-1 or other MAP65 proteins from microtubules.

### Nucleic Acids and Polypeptides

As used herein, nucleic acids can include DNA and RNA, and includes nucleic acids that contain one or more nucleotide analogs or backbone modifications. A nucleic acid can be single stranded or double stranded, which usually depends upon its intended use.

Nucleic acids are provided herein (e.g., SEQ ID NOs: 1 and 3) that encode for MAP65-1 proteins (used interchangeably with polypeptides) from Arabidopsis (SEQ ID NO:2) or from Glycine max (SEQ ID NO:4), respectively. Also provided are nucleic acids and proteins that differ from SEQ ID NOs: 1 and 3 and SEQ ID NOs: 2 and 4, respectively. Nucleic acids and proteins that differ in sequence from SEQ ID NOs: 1 and 3 and SEQ ID NOs: 2 and 4 can have at least 80% sequence identity (e.g., at least 85%, 90%, 95%, or 99% sequence identity) to SEQ ID NOs: 1 and 3 and SEQ ID NOs: 2 and 4, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

It would be understood that fragments of SEQ ID NO: 1 or 3 are suitable for use in the methods described herein. Nucleic acid fragments suitable for use are those fragments that encode a polypeptide having the functional activity described herein. These fragments can be called “functional fragments,” although it is understood that it is not the nucleic acid that possesses functionality.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a construct, or an expression vector) for convenience of manipulation or to generate a fusion protein, discussed in more detail below. In addition, an isolated nucleic acid molecule can include an engineered nucleic acid molecule such as a recombinant or a synthetic nucleic acid molecule.

As used herein, a “purified” protein is a protein that has been separated or purified from cellular components that naturally accompany it. Typically, the protein is considered “purified” when it is at least 70% (e.g., at least 75%, 80%, 85%, 90%, 95%, or 99%) by dry weight, free from the proteins and naturally occurring molecules with which it is naturally associated. Since a protein that is chemically synthesized is, by nature, separated from the components that naturally accompany it, a synthetic protein is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Proteins can be purified from natural sources (e.g., a biological sample) by known methods such as DEAE ion exchange, gel filtration, and hydroxyapatite chromatography. A protein also can be purified, for example, by expressing a nucleic acid in an expression vector. In addition, a purified protein can be obtained by chemical synthesis. The extent of purity of a protein can be measured using any appropriate method, e.g., column chromatography, polyacrylamide gel electrophoresis, or HPLC analysis.

If desired, changes can be introduced into a nucleic acid molecule, thereby leading to changes in the amino acid sequence of the encoded protein. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A conservative amino acid substitution is one in which one amino acid residue is replaced with a different amino acid residue having a similar side chain (see, for example, Dayhoff et al. (1978, in Atlas of Protein Sequence and Structure, 5 (Suppl. 3): 345-352), which provides frequency tables for amino acid substitutions), and a non-conservative substitution is one in which an amino acid residue is replaced with an amino acid residue that does not have a similar side chain.

A construct containing a nucleic acid (e.g., a nucleic acid that encodes a protein) also is provided. Constructs, including expression constructs, are commercially available or can be produced by recombinant DNA techniques routine in the art. A construct containing a nucleic acid can have expression elements operably linked to such a nucleic acid, and further can include sequences such as those encoding a selectable marker (e.g., an antibiotic resistance gene). A construct containing a nucleic acid can encode a chimeric or fusion protein (i.e., a first protein linked to a second, usually heterologous to the first, protein, which can be at either the N-terminus or C-terminus of the first protein). Representative second heterologous proteins are those that can be used in purification of the encoded first protein (e.g., 6×His tag, glutathione S-transferase (GST)).

Expression elements include nucleic acid sequences that direct and regulate expression of nucleic acid coding sequences. One example of an expression element is a promoter sequence that drives expression of a coding sequence. Expression elements also can include introns, enhancer sequences, response elements, or inducible elements that modulate expression of a nucleic acid and target sequences that determine where the polypeptide is located within the cell, within the membrane, or outside of the cell. Expression elements can be of bacterial, yeast, insect, mammalian, or viral origin, and constructs can contain a combination of elements from different origins. As used herein, operably linked means that a promoter or other expression element(s) are positioned in a construct relative to a nucleic acid in such a way as to direct or regulate expression of the nucleic acid. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Constructs as described herein can be introduced into a cell (e.g., a host cell). As used herein, “host cell” refers to the particular cell into which the nucleic acid is introduced and also includes the progeny or potential progeny of such a cell. A host cell can be any cell (e.g., a prokaryotic or eukaryotic cell). For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be appreciated that, although any number of hybridization and washing conditions can be used to examine hybridization of a probe nucleic acid molecule to a plurality of immobilized target nucleic acids, it is more important to examine hybridization of a probe to the plurality of target nucleic acids under identical hybridization, washing, and exposure conditions. Preferably, the plurality of target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### Transgenic Plants and Methods of Making

The sequences described herein can be overexpressed in plants. Transgenic plants are provided that are transformed with a nucleic acid molecule described herein (e.g., SEQ ID NOs: 1 or 3) or a portion thereof (e.g., encoding a functional fragment) under control of a promoter that is able to drive expression in plants (e.g., a plant promoter, e.g., 35S or ubiquitin from soybean). As discussed herein, a nucleic acid molecule used in a plant expression vector can have a different sequence than a sequence described herein, which can be expressed as a percent sequence identity (e.g., relative to SEQ ID NOs: 1 or 3) or based on the conditions under which sequences hybridize (e.g., to SEQ ID NOs: 1 or 3). As an alternative to using a full-length sequence, a portion of the sequence can be used that encodes a polypeptide fragment having the desired functionality (referred to herein as a “functional fragment”). When used with respect to nucleic acids, it would be appreciated that it is not the nucleic acid fragment that possesses functionality but the encoded polypeptide fragment.

Methods of introducing a nucleic acid (e.g., an exogenous nucleic acid) into plant cells are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2 (7): 1565-72)), liposome-mediated DNA uptake, or electroporation. Following transformation, the transgenic plant cells can be regenerated into transgenic plants. As described herein, expression of the transgene results in plants that exhibit resistance to Pseudomonas syringae pv. Glycinea and/or Phytophthora sojae. The regenerated transgenic plants can be screened for resistance to the indicated pathogens, compared to a corresponding plant lacking the exogenous nucleic acid, and can be selected for use in, for example, a breeding program as described herein.

Hybrids, varieties, lines, or cultivars are provided that contain an exogenous nucleic acid encoding a MAP65-1 protein (e.g., SEQ ID NOs: 1 or 3 encoding SEQ ID NOs: 2 or 4, respectively). As described herein, plants expressing an exogenous MAP65-1 nucleic acid (e.g., SEQ ID NOs: 1 or 3) can exhibit resistance to the indicated pathogen, compared to a corresponding plant lacking the exogenous nucleic acid.

A plant carrying a construct expressing an exogenous MAP65-1 nucleic acid can be used in a plant breeding program to create novel and useful cultivars, lines, varieties and hybrids. Thus, in some embodiments, a T₁, T₂, T₃ or later generation plant containing an exogenous MAP65-1 nucleic acid is crossed with a second plant, and progeny of the cross are identified in which the exogenous MAP65-1 nucleic acid is present. It will be appreciated that the second plant can contain the same exogenous nucleic acid as the plant to which it is crossed, a different nucleic acid, a mutation, or wild type plant. Additionally or alternatively, a second line can exhibit a phenotypic trait such as, for example, disease resistance, high yield, leaf quality, height, plant maturation, stalk size, and/or leaf number per plant.

Breeding is carried out using known procedures. DNA fingerprinting, SNP or similar technologies may be used in a marker-assisted breeding program to transfer or breed desired alleles into other lines, varieties or cultivars, as described herein. Progeny of the cross can be screened for an exogenous nucleic acid using methods described herein, and plants having an exogenous nucleic acid disclosed herein (e.g., SEQ ID NOs: 1 or 3) can be selected. For example, plants in the F₂ or backcross generations can be screened using a marker developed from a sequence described herein or a fragment thereof, using one of the techniques listed herein. Plants also can be screened for resistance to the indicated pathogens, and those plants having one or more of such phenotypes, compared to a corresponding plant that lacks the exogenous nucleic acid, can be selected. Plants identified as possessing the exogenous nucleic acid and/or the desired phenotype can be backcrossed or self-pollinated to create a second population to be screened. Backcrossing or other breeding procedures can be repeated until the desired phenotype of the recurrent parent is recovered.

Successful crosses yield F₁ plants that are fertile and that can be backcrossed with one of the parents if desired. In some embodiments, a plant population in the F₂ generation is screened for the exogenous nucleic acid using standard methods (e.g., PCR with primers based upon the nucleic acid sequences disclosed herein). Selected plants are then crossed with one of the parents and the first backcross (BC₁) generation plants are self-pollinated to produce a BC₁F₂ population that is again screened for the presence of exogenous nucleic acid. The process of backcrossing, self-pollination, and screening is repeated, for example, at least four times until the final screening produces a plant that is fertile and reasonably similar to the recurrent parent. This plant, if desired, is self-pollinated and the progeny are subsequently screened again to confirm that the plant contains the exogenous nucleic acid and exhibits the desired phenotype. Breeder's seed of the selected plant can be produced using standard methods including, for example, field testing, genetic analysis, and/or confirmation of the phenotype.

The result of a plant breeding program using the plants described herein are novel and useful cultivars, varieties, lines, and hybrids. As used herein, the term “variety” refers to a population of plants that share constant characteristics which separate them from other plants of the same species. A variety is often, although not always, sold commercially. While possessing one or more distinctive traits, a variety is further characterized by a very small overall variation between individual with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

Depending on the plant, hybrids can be produced by preventing self-pollination of female parent plants (i.e., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), nuclear male sterility, genetic male sterility, molecular male sterility wherein a transgene inhibits microsporogenesis and/or pollen formation, or self-incompatibility. Female parent plants containing CMS are particularly useful. In embodiments in which the female parent plants are CMS, the male parent plants typically contain a fertility restorer gene to ensure that the F₁ hybrids are fertile. In other embodiments in which the female parents are CMS, male parents can be used that do not contain a fertility restorer. F₁ hybrids produced from such parents are male sterile. Male sterile hybrid seed can be interplanted with male fertile seed to provide pollen for seed-set on the resulting male sterile plants.

Varieties, lines and cultivars described herein can be used to form single-cross F₁ hybrids. In such embodiments, the plants of the parent varieties can be grown as substantially homogeneous adjoining populations to facilitate natural cross-pollination from the male parent plants to the female parent plants. The F₂ seed formed on the female parent plants is selectively harvested by conventional means. One also can grow the two parent plant varieties in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent. As another alternative, double-cross hybrids can be created wherein the F₁ progeny of two different single-crosses are themselves crossed. Self-incompatibility can be used to particular advantage to prevent self-pollination of female parents when forming a double-cross hybrid.

As used herein, an “increase” in pathogen resistance refers to an increase (e.g., a statistically significant increase) in resistance by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to corresponding pathogen resistance exhibited by a corresponding plant lacking the exogenous nucleic acid grown under corresponding conditions. An increase in resistance can be exhibited as an increase in survival and/or an increase in health of the pathogen-challenged plant. As used herein, statistical significance refers to a p-value of less than 0.05, e.g., a p-value of less than 0.025 or a p-value of less than 0.01, using an appropriate measure of statistical significance, e.g., a one-tailed two sample t-test.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Bioinformatics Tools

The MAP65 protein sequences of both Arabidopsis and soybean were retrieved from Phytozome (phytozome-next.jgi.doe.gov). A maximum likelihood phylogenic tree was built with Molecular Evolutionary Genetics Analysis (MEGA) 11 with 2000 bootstraps. A distantly related MAP65-like protein from Chlamydomonas reinhardtii (CRE14. g614050) was included as an outgroup as previously reported.

### Example 2—Assembly of Binary Vectors for Soybean Transformation

AtMAP65-1 (At.05G55230) or GmMAP65-1 (Glyma.02g295100) were PCR-amplified with high fidelity polymerase Pfu (Stratagene Cat #600380) from cDNAs of Arabidopsis and soybean variety Thorne, respectively. The PCR products were cloned into pENTR-D TOPO vector (Invitrogen Cat #K240020). The resulting pENTR constructs were recombined with destination vector pLN6006 containing a cauliflower mosaic virus 35S promoter by LR reactions resulting in pLN6013 (35S-A/MAP65-1-HA) or pLN6015 (35S-GmMAP65-1-HA), respectively. pLN6013 and pLN6015 were then mobilized into A. tumefaciens strain EHA101 via triparental mating. The strains and constructs used in this study are listed in Table 1.

TABLE 1
Plasmids and Strains Used in this Study

| | | Reference or |
|---|---|---|
| Strain or Plasmid | Characteristics | source |
| A. tumefaciens EHA101 | C58 (Rif [symbol] ) Ti pEHA101 (pTiBo542 D T-DNA) (Km [symbol] ) | (Hood et al., |
| | Nopaline | 1986) |
| E. coli DB3.1 | F [symbol] gyrA462 endA1 Δ(sr1-recA) mcrB mrr hsdS20 (rₐ [symbol] , | Invitrogen |
| | mₐ [symbol] ) supE44 ara-14 galK2 lacY1 proA2 rpsL20 (Sm [symbol] ) | |
| | xyl-5 λ [symbol] leu mtl-1 | |
| E. coli DH5α | supE44 ΔlacU169(φ80lacZΔM15) hsdR17 recA1 endA1 | (Hanahan, 1883) |
| | gyrA96 thi-1 relA1, Nal [symbol] | |
| Pseudomonas syringae | Wild type; spontaneous Rif [symbol] | This work |
| pv. glycinea LN10 | | |
| Phytophthora sojae | Wild type; spontaneous Rif [symbol] Pi [symbol] Ap [symbol] | Wenbo Ma |
| 6497 | | |
| Phytophthora sojae | Wild type; spontaneous Rif [symbol] Pi [symbol] Ap [symbol] | Loren Giesler |
| NE21 | | |
| pENTR/D-TOPO | Gateway system donor vector, Km [symbol] | Invitrogen |
| pLN5010 | pENTR derivative carrying AtMAP65-1 Km [symbol] | (Guo et al., 2016) |
| pLN6006 | pPTN200 Gateway destination binary vector with 36S | This work |
| | promoter and HA fusion at C-terminus, Sp [symbol] Cm [symbol] | |
| pLN6011 | pENTR derivative carrying GmMAP65-1, Km [symbol] | This work |
| pLN6013 | PLN6006 derivative carrying AtMAP65-1, Sp [symbol] | This work |
| pLN6015 | pLN6006 derivative carrying GmMAP65-1, Sp [symbol] | This work |
| pPTN200 | Soybean binary vector, Sp [symbol] | This work |

[symbol]

indicates data missing or illegible when filed

### Example 3—Plant Materials

All the soybean plants were grown in a standard greenhouse environment. Transformations of wild type soybean variety Thorne were performed with A. tumefaciens strain EHA101 strains harboring AtMAP65-1-HA and GmMAP65-1-HA following a protocol previously described. Derived primary events established in the greenhouse were screened for expression of the selection marker, bar gene encoding for phosphinothricin acetyltransferase (PAT) that detoxifies the herbicide glufosinate, with a leaf painting technique. Briefly, a solution of the glufosinate (200 mg/L) was applied to leaves with a cotton swab to the adaxial surface of true leaflets of 3-week-old plants. The glufosinate tolerant plants were grown to maturity. Inheritance of the transgenic alleles were tracked using the leaf painting assay and expression of the gene of interest alleles monitored via immunoblots, using anti-HA antibodies (Roche).

### Example 4—Bacterial Growth Assays

Bacterial growth assays with soybean plants were carried out as previously described. Briefly, overnight cultures of P. syringae glycinea LN10 strain were grown on King's B (KB) plates and then were resuspended to a cell density of 10⁵ cells/ml with sterile water. Four-week-old soybean plants at the V3 developmental stage (with two trifoliolate leaves) were vacuum-infiltrated with bacterial suspension containing 0.02% Silwet L-77™ (Lehle Seeds). Leaf disks of 0.3 cm in diameter were punched at indicated times (0 and 3 days) post inoculation. Samples were ground in sterile distilled water, and 10-fold serial dilutions were plated on KB agar plates. The number of bacteria in the soybean leaf tissue were determined by counting colonies on the plates after incubation for 3 days at 30° C.

### Example 5—Pathogenicity Assay with Phytophthora sojae

Briefly, overnight cultures of Phytophthora sojae strains were grown on 10% V8 agar containing 10 μg/ml pimaricin, 25 μg/ml rifampicin, and 50 μg/ml ampicillin in the dark at 25° C. until the growth reaches edge of the plates. Five discs of 0.3 cm from periphery of growing culture and place in a fresh sterile petri dish with 10% V8 liquid medium. The discs were kept at 25° C. in the dark overnight. V8 medium was discarded and the petri dish was flooded with 15 ml sterile Petri's solution (0.25 mM CaCl₂), 1 mM MgSO₄, 1 mM KH₂PO₄, 0.8 mM KCl) and let sit 30 min at 25° C. in the dark. After wash for 5 times, ten ml sterile Petri's Solution were added and incubated for 4-18 hours at 20° C. in the dark. After 4 hours, sporangia should be visible under a microscope with 40× magnification. The petri dish was placed in 4° C. for 30 min when it was ready to release spores. The plates were returned to room temperature for 30 minutes. The zoospores were carefully resuspended by swirling the plate and then collected bypass the resuspension through a sterile cell mesh (40 μm). The zoospore concentration was measured using a hemocytometer and adjusted to 20,000 zoospores per ml for future inoculation. Soybean plants were grown in dark at 25° C. for 5 days. A small 1 cm incision was made about 2 cm down from the bend in the hypocotyl. One hundred zoospores were inoculated onto the incision. Two days after inoculation, a 2 cm region centered on the incision site was detached for imaging or grinded to powder in liquid nitrogen for DNA extraction.

### Example 6—Quantitative Digital Droplet PCR (ddPCR) Analyses

The copy number of PsACT1 gene in P. sojae strains were quantified with ddPCR using a Bio-Rad QX200 Digital droplet PCR system. Briefly, 5-day-old seedling plants were infected with 100 zoospores of P. sojae Ps6497 or PsNE-21 and sampled 48 h after inoculation. Total genomic DNA was extracted with DNA extraction reagent (200 mM Tris, pH 8, 200 mM NaCl, 25 mM EDTA pH 8, 2% SDS, 100 μg/ml RNase A) and used as templates for quantitative ddPCR. The gene-specific primers used in ddPCR were listed in Table 2.

TABLE 2
Primers Used for Cloning and qPCR

| Primer | Construct/ | |
|---|---|---|
| Name | gene | Sequence |
| P4538 | PLN5010 | 5′-CACCGAATTCATGGCAGTTAC |
| | | AGATACTGAAAG-3′ |
| P4539 | | 5′-TGGTGAAGCTGGAACTTGATG-3′ |
| P4671 | HA | 5′-TCAAGCGTAATCTGGAACATCG-3′ |
| P5817 | PLN6011 | 5′-CACCATGGCAGTGACCGAAGCT-3′ |
| P5818 | | 5′-GGGTGATGCCGGGATGGGTTCAGT-3′ |
| P6667 | PSACT1 | 5′-ACTGCACCTTCCAGACCATC-3′ |
| P6668 | | 5′-CCACCACCTTGATCTTCATG-3′ |

### Example 7—Pathogenicity Assay with Heterodera glycines

SCN bioassays were conducted following the modified protocol previously described. Thorne, AtMAP65-1 and GmMAP65-1 transgenic plants, and Williams 82 as a susceptible control were inoculated with H. glycines. Soybean plants were grown in a greenhouse maintained at 27° C. with 16 h of light per day. Two days after transplanting, the roots of each individual plant were inoculated with 200 H. glycines eggs and grown for 30 days. After 30 days, the plant height was measured, and cysts were collected. Cysts were broken to release eggs, and the number of eggs was counted. The reproductive factor (Rf) of SCN was determined by dividing the total number of eggs produced per pot by the number inoculated. Data were based on two independent biological replicate experiments (n>20 roots/independent lines).

### Example 8—Oryzalin Tolerance Assay

Wild type soybean Thorne, AtMAP65-1, and GmMAP65-1 plants were germinated and transferred on 0 μM (Mock), 1 μM, 100 μM, and 1,000 μM oryzalin plates. Pictures were taken at 10-day-old seedling plants. Soybean plants were grown in the growth chamber and treated with water containing various amount oryzalin for 3 weeks (Surflan® 40.4% oryzalin). Two herbicide tolerance assessments were monitored under greenhouse conditions, leaf damage at emergence stage, following a preemergent application of Surflan® equivalent to 0, 2, 4 and 8 lb ai/acre and leaf damage at 2 weeks post application of Surflan® over the top on V3 stage plants, using the same rates.

### Example 9—Low Temperature Stress Assay

For low temperature response experiment, the AtMAP65-1 and GmMAP65-1, and wild type Thorne plants were planted in PRO-MIX potting soil (Premier Horticulture, Inc) and grown in a growth chamber (16 h day/8 h night) at 24° C. for 7 days. One-week-old plants were transferred to 4° C. or 24° C. growth chambers and maintained for 10 more days till harvesting for biomass analyses. The fresh and dry weights were measured following a previous method with minor modification. For dry weight, samples were oven-dried at 65° C. for 48 h and measured.

### Example 10—Ion Leakage Assay

Three-week-old plants were grown in a growth chamber with 16 h of light per day (29° C./23° C. day/night). Leaf disks of 0.8 cm in diameter were punched from the second true leaf, which floated in 3 mL of water. Samples were cooled precisely using a refrigerated circulator (AP15R-40, VWR). Following a 30 min equilibration at 0° C., samples were cooled to −1° C. for over 30 minutes, ice formation was nucleated by addition of a purified water ice chip, then cooling continued at the rate of −1° C./h. Samples were collected at temperatures from 0° C. to −5° C. The chilled leaf samples were incubated in a 4° C. water bath for an hour. The temperature was raised to room temperature for 30 min and the samples were mixed at 250 rotations per min for 15 min. Conductivity of the samples was measured by an ORION STAR A212 conductivity meter. For total conductivity measurements, samples were heated at 65° C. for 30 min and then cooled to room temperature and mixed and measured as above. To calculate the leakage, the percentage relative to total ion leakage was calculated and plotted.

### Example 11—Statistical Analyses of Datasets

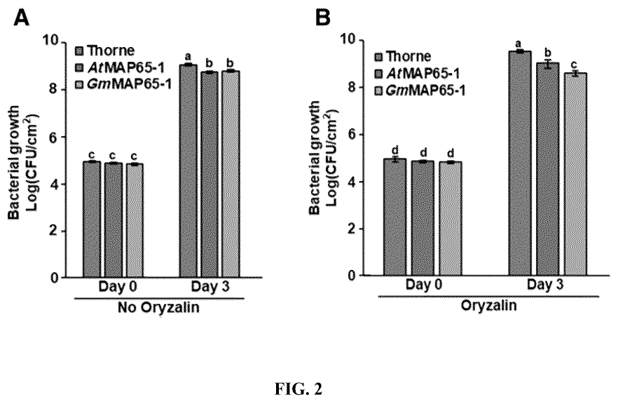

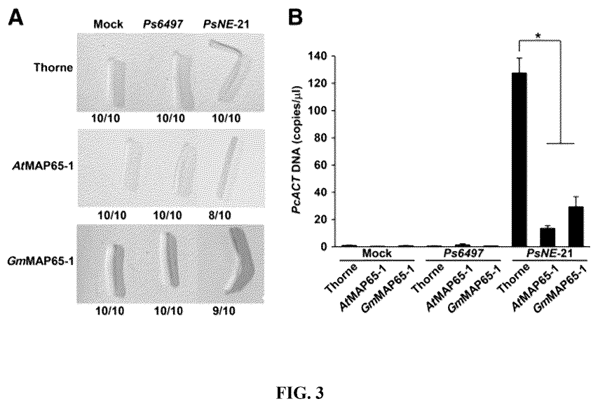

Datasets were analyzed via ANOVA and mean differences separated by LSD (FIGS. 2, 4, 5 and 9) or student t-test (FIG. 3). All experiments were repeated 2-3 times, with each figure displaying the combined results of the experiments.

### Example 12—Soybean MAP65-1 Transgenic Plants Exhibit Resistance to P. syringae pv. glycinea LN10

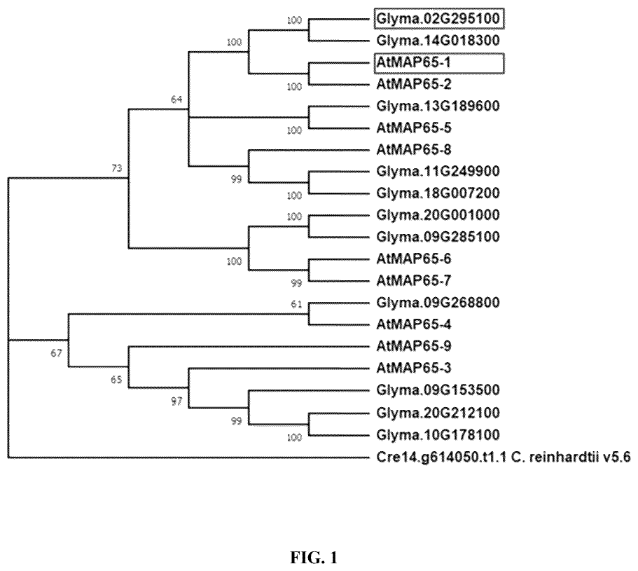

Soybean genome (Glycine max var. Williamson 82) contains 11 genes annotated as MAP65 homologs located across eight different chromosomes. These MAP65 gene models are highly homologous to those from Arabidopsis, with Glyma02g295100 (GmMAP65-1) being the closest one to the Arabidopsis AtMAP65-1 (At5g55230), which shares 79% identity at the amino acid level (FIG. 1). The AtMAP65-1 is targeted by P. syringae effector HopE1 to facilitate pathogenesis, suggesting it is an important component of plant immunity.

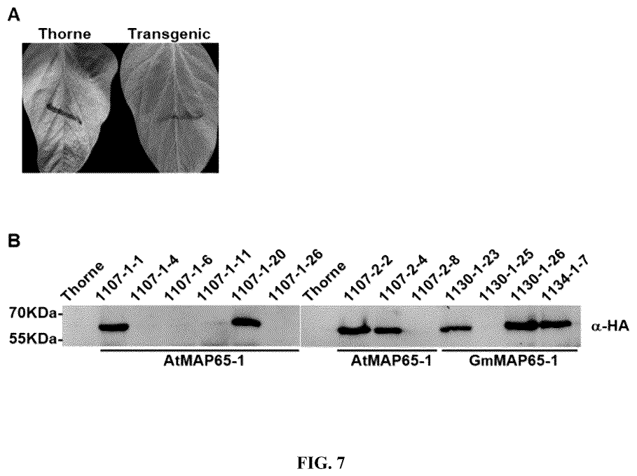

To determine if enhanced expression of MAP65-1 proteins can contribute to resistance of soybean to pathogen challenge, a set of transgenic soybean events were developed from the wild type G. max var. genotype Thorne. These events constitutively express either AtMAP65-1 or GmMAP54-1, both fused with an epitope HA tag at the carboxyl terminus, each of which were phenotyped in multiple pathogen assays. Homozygous T₃ generation lineages of AtMAP65-1 (events 1107 Jan. 1, 1107 Jan. 20, 1107 Feb. 2, and 1107 Feb. 4) and GmMAP65-1 (events 1130 Jan. 23, 1130 Jan. 26, and 1134 Jan. 7) soybean were identified using a leaf painting assay for the plant selectable marker bar gene (FIG. 7A) and the expression of the MAP65-1 transgenic alleles were confirmed with immunoblot analysis using HA antibody (FIG. 7B).

To determine if a biotic resistance phenotype is imparted by elevated MAP65-1 levels in soybean, both AtMAP65-1 (event 1107 Jan. 1) and GmMAP65-1 (event 1130 Jan. 26) transgenic soybean were challenged with the bacterial pathogen, P. syringae pv. glycinea (Pgy) strain LN10, a virulent strain in soybean plants. The growth of Pgy LN10 in both AtMAP65-1 and GmMAP65-1 transgenic soybean events was significantly reduced relative to wild type Thorne (FIG. 2A). When the plants were treated with 100 μM oryzalin, an herbicide that inhibit microtubule polymerization, and subsequently challenged with the pathogen, similar to plants without oryzalin treatment, both events also exhibited enhanced resistance response relative to the control plants. On the other hand, the GmMAP65-1 soybean maintained the resistance response, following the oryzalin application, and had further reduced bacterial growth comparing to AtMAP65 plants (FIG. 2B).

### Example 13—Enhanced Expression of MAP65-1 Elevates Resistance to the Oomycete Pathogen Phytophthora sojae but Not a Pathogenic Nematode Heterodera glycines

To determine if constitutively-expressed transgenic MAP65-1 alleles in soybean confers a broader resistance to other phytopathogens, the soybean events were challenged with Phytophthora sojae, a stem and root rot pathogen, and Heterodera glycines, the soybean cyst nematode (SCN).

Soybean seedlings were grown in the dark, and hypocotyls were inoculated with two P. sojae strains, PsNE21 and Ps6497. Control, wild type Thorne, displayed a susceptible phenotype to the P. sojae strain PsNE21, that was apparent from the bent hypocotyls observed 48 hours after inoculation, but no susceptible phenotype was seen in control plants challenged with Ps6497 strain (FIG. 3A). The latter observation agrees with the resistance response observed upon a Ps6497 challenge on genotype W82, which like Thorne, carries the Rsp1k resistance allele. However, both AtMAP65-1 and GmMAP65-1 soybean events exhibited significantly less bent hypocotyl phenotype at the 48 hours post challenge time point with the P. sojae NE21 (PsNE-21) strain challenge compared with wild type Thorne (FIG. 3A). Relative biomass of P. sojae strains were estimated by quantitative digital droplet PCR (ddPCR) (BioRad QX600 Droplet Digital PCR) that determine the abundance of P. sojae PsACT1 of genomic DNA extracted from infected soybean hypocotyls. Consistent with the visible reduction in hypocotyl bending phenotype, the abundance of P. sojae PsACT1 was drastically reduced in soybean events expressing the transgenic AtMAP65-1 and GmMAP65-1 alleles suggesting accumulated biomass of PsNE-21 was significantly less than that accumulated in wild type Thorne (FIG. 3B).

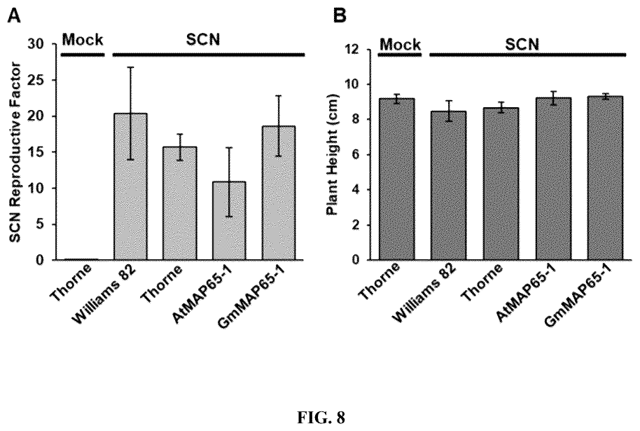

However, no significant differences were observed in response to H. glycines challenge of the either the AtMAP65-1 or GmMAP65-1 soybean events, as reflected by the number of eggs produced in infected roots, a metric for H. glycines tolerance (FIG. 8). These results indicate that constitutive expression of the MAP65-1 transgenic alleles in soybean can impact a resistance phenotype to the virulent P. sojae strain PsNE-21 but not H. glycines.

### Example 14—Constitutive Expression of MAP65-1 Transgenic Alleles Increases the Tolerance of Soybean to the Herbicide Oryzalin

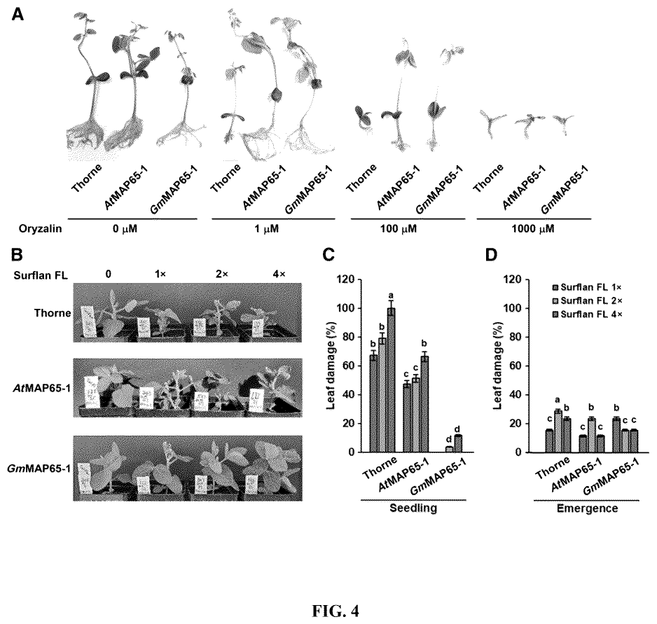

The effects of constitutive expression of AtMAP65-1 and GmMAP65-1 transgenic alleles on the tolerance to the herbicide oryzalin was also assessed. Here, seeds were sown on solid MS medium supplemented with 0 μM (Mock), 1 μM, 100 μM, and 1,000 μM oryzalin (FIG. 4A). Seeds derived from the AtMAP65-1 and GmMAP65-1 soybean events exhibited superior seedling development, both roots and hypocotyls, sown on oryzalin supplemented medium, at levels of 1 μM or 100 μM relative to wild type Thorne, however, the growth of all soybean seedlings was severely arrested at a concentration of 1,000 μM. (FIG. 4A). Control seedlings germinating under oryzalin pressure were highly distorted in hypocotyl length and impaired in root growth at concentrations from 1 μM (FIG. 4A).

The AtMAP65-1 and GmMAP65-1 soybean events were also assessed for an herbicide tolerance phenotype under greenhouse conditions. Here, Surflan® (40.4% a.i. oryzalin), a commercial oryzalin formulation, was applied. Thorne and the respective, MAP65-1 soybean events were treated by soil application of Surflan® at different concentrations. The soybean events expressing AtMAP65-1 and GmMAP65-1 transgenic alleles showed significant resistance to oryzalin applications at different concentrations relative to controls Thorne (FIG. 4B). When treated with Surflan®, the leaf damages of AtMAP65-1 and GmMAP65-1 transgenic plants were much less severe than wild type Thorne at seedling stage (FIG. 4C), while the magnitudes in difference were less prominent after emergence (FIG. 4D). The herbicide tolerance assays, both greenhouse, and in vitro germination, reflect the ability of enhancing the levels of MAP65-1 in plant cells, can impart an herbicide tolerance phenotype to oryzalin in soybean.

### Example 15—Expression of the Transgenic MAP65-1 Alleles Elevate Cold Tolerance in Soybean

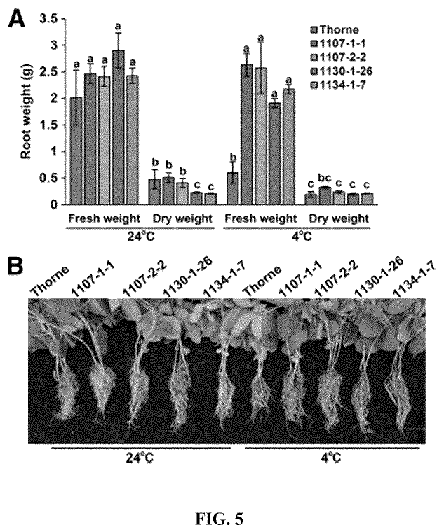

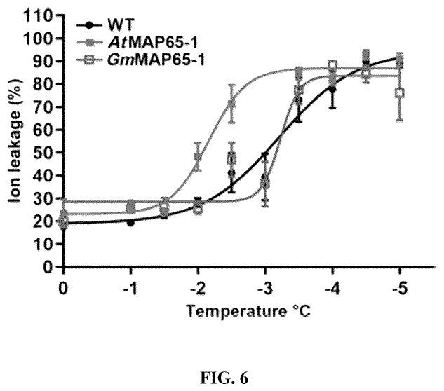

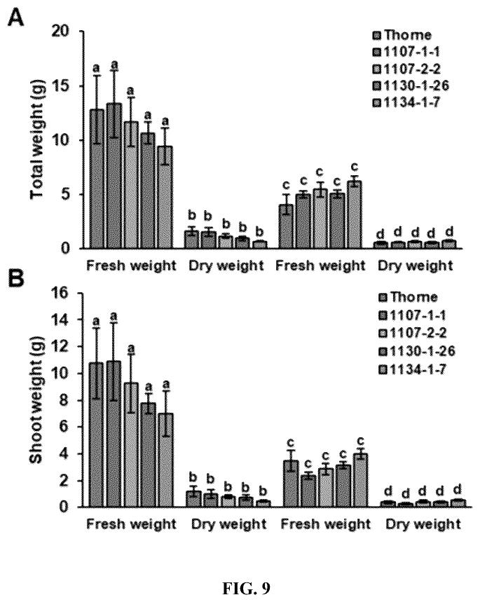

MAP65-1 proteins have been shown to stabilize microtubule bundles against a low temperature stress challenge in both animal and plants systems. Thus, it is logical to hypothesize that an increase in MAP65-1 levels in soybean may translate to a tolerance to a cold stress challenge. To test this, we chilled plants at 4° C. for 10 days. Cold treated AtMAP65-1 (events 1107-1-1 and 1107-2-2 (SFig1)) and GmMAP65-1 (events 1130-1-26 and 1134-1-7) events exhibited significantly increased fresh root biomass compared to the wild type Thorne plants (FIG. 5A-B), while it was not significantly different in total fresh weight, weight of aerial parts, or dry weights (FIG. 9A-9B). Additionally, the AtMAP65-1 and GmMAP65-1 soybean events, along with wild type Thorne plants, were subjected to a freezing challenge and assayed for ion leakage, an indicator for cytosol leakage resulting from cold-associated damages of plant cell membranes, which is a predictor for cold tolerance. A slight change in percentages of ion leakage were detected in GmMAP65-1 event, with a lagged shift from −2 and −4° C. (FIGS. 6A and 6C), while a slightly reduced ion leakage was seen for AtMAP65-1 transgenic event (FIG. 6B), suggesting diverse contribution with different loci and that the ectopic expression of GmMAP65-1 may improve the freezing tolerance in soybean.

### Example 16—Interpretation

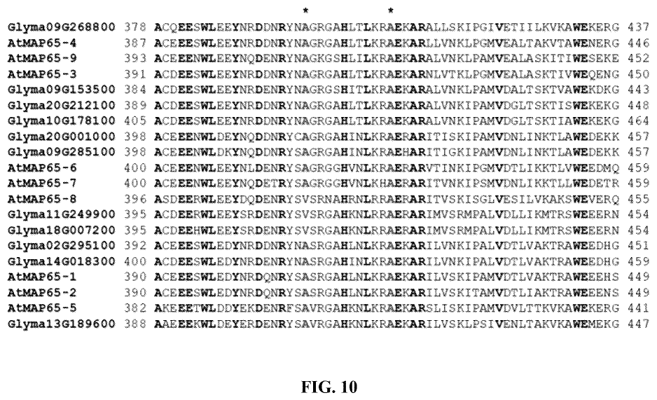

Studies of MAP65 revealed that family members are highly similar at the amino acid level, with the greatest variation residing in the C-terminal region of the protein. Within the variable C-terminal region, two conserved residues, Ala409 and Ala420, are required for MAP65-1 ability to interact with microtubules and impact its formation of functional dimers (FIG. 10). This region is also a site of post-translational regulation via phosphorylation. Wherein, the phosphorylated protein is mitigated in its ability to bind microtubules.

Constitutive expression cassettes were assembled and introduced into soybean. A subset of the derived transgenic events was evaluated in a series of assays investigating changes in resistance responses to both biotic and abiotic stresses.

The gene models selected for soybean transformation were AtMAP65-1 (At05G55230) and GmMAP65-1 (Glyma.02G295100). A sequence alignment of the C-terminal region, composed of the last 50 amino acid residues of the nine Arabidopsis and eleven soybean MAP65 family members, is shown in FIG. 10. AtMAP65-1 and GmMAP65-1 are 79% identical across the entire protein, while between the most variable ‘FR4’ fragment, share 66% identity, with the remaining regions sharing 84% (aa340-494), 84% (aa151-339), and 77% (aa1-150). Both gene models contain the conserved Ala409 and Ala420 residues, with only one Arabidopsis member, AtMAP65-8 and two soybean members (Glyma. 11G249900 and Glyma.18G007200) harboring the Ala409Val change that translates to a MAP65-1 null for its ability to bind and cross link microtubules (FIG. 10).

Maintaining cellular microtubule integrity and proper dynamics during plant growth and development is vital and plays a major role in protection of against biotic and abiotic challenges. Soybean events with enhanced levels of MAP65-1, either GmMAP65-1 or AtMAP65-1, displayed a strong tolerance phenotype to application of herbicide molecule oryzalin, whose mode of action is via disruption of microtubule integrity (FIG. 4). This outcome reflects the improved stability of microtubules in the respective events manifested by the constitutive expression of the respective transgenic MAP65-1 alleles. The herbicide tolerance phenotype was observed both in vitro culture assay, sowing seeds on medium supplemented with the herbicide (FIG. 4A), and application of the herbicide at approximately V3 stage of development under greenhouse conditions (FIG. 4B). However, an herbicide tolerance phenotype, while statistically significant at certain preemergent rates tested, the outcome was as not dramatic relative to the post-emergence application at V3 stage (FIG. 4B). This differential tolerance outcome under the greenhouse environment assay may be related to the timing of the assessments. In the preemergent assay observations were taken at a stage when the first true leaf is emerging (VC/V1 stage), while the over-the-top application at V3 stage of development was assessed two weeks post application. Given the former is observing damage on tissues consisting of preformed unifoliate leaves and first leaf primordia in the embryo, where during germination there is a combination of expansion of cells and mitotic cell lineages, while the latter observation time point reflected tissues derived primarily from cell division, with some expansion.

The herbicide tolerance phenotype observed in the in vitro assay, was similar between the AtMAP65-1 and GmMAP65-1 transgenic alleles (FIG. 4A). However, ectopic expression of GmMAP65-1 displayed a superior tolerance in the greenhouse assay relative to that imparted by the AtMAP65-1 transgenic allele. These results suggest that homodimers of MAP65-1 have superior microtubule binding ability, and the presence of putative heterodimers between AtMAP65-1 and GmMAP65-1 have reduced binding capacity, thus mitigating microtubule crosslinking by a putative heterodimer molecule.

The enhanced microtubule stability outcome, as reflected by the observed herbicide tolerance phenotype, was assessed for protection against three soybean pathogen challenges, Phytophthora sojae, P. syringae pv. glycinea LN10 and soybean cyst nematode H. glycines. The results displayed an enhanced tolerance to both bacterial (FIGS. 2A and 2B) and oomycete pathogens (FIG. 3), but not the soybean cyst nematode H. glycines (FIG. 8).

In the nematode/plant pathosystem southern root-nematode (Meloidogyne incognita), successful pathogenesis leads to the formation of ‘giant cells’. These are large multinucleated cell feeding sites for the nematode, that are induced by the pathogen, and are the result of multiple rounds of cell division, devoid of completion of cytokinesis. H. glycines also triggers multinucleated cell structures during their interaction with the host, designated syncytial cells. The development of both giant and syncytial cells is associated with depolymerization of microtubules. The formation of giant cells has a direct link with the MAP65 family member AtMAP65-3, via its role in proper network array of microtubules during mitosis. Arabidopsis lines devoid of AtMAP65-3, display multiple developmental phenotypes, but are resistant to root-knot nematode infection. Hence, proper microtubule network establishment plays a critical role in plant/nematode interaction but enhancing stability of microtubule network through elevated levels of MAP65-1 does not appear to be a factor in the interaction with the soybean cyst nematode and its host (FIG. 8).

Pathogen challenges with bacterial and oomycete pests, on the other hand, suggest a direct link between microtubule stability and a resistance response for these pests. In the P. syringae pv. glycinea LN10 challenge assay, when the pathogen challenge was applied post-oryzalin application, an enhanced disease state was observed in wild type control, supporting the link between microtubule stability and disease status. In the soybean events with enhanced levels of MAP65-1, a significant reduction in bacterial growth was observed, with the GmMAP65-1 event, displaying high degree of resistance, in the post-oryzalin challenge (FIG. 2B), which agrees with the herbicide tolerance outcome under greenhouse conditions, which ectopic expression of MAP65-1 translates to a higher microtubule stability as compared to expression of the Arabidopsis allele (FIG. 4B).

The outcome of oomycete challenge assays also suggests enhanced microtubule stability can translate to a resistance response, as indicated by reduction in both visual (FIG. 3A) and pathogen biomass (FIG. 3B) in hypocotyl segments from soybean events with enhanced MAP65-1 levels, but, here, the phenotypic outcomes were similar with AtMAP65-1 and GmMAP65-1 transgenic alleles.

The relationship between microtubule stability and abiotic stress in plants is well documented, where MAP65-1 has been associated with tolerance to low temperature and salt. When the soybean events were assessed in a freezing assay, monitoring ion leakage in leaf discs, only slight, yet not significant, positive trend was observed in the GmMAP65-1 event (FIG. 6). However, a low temperature challenge study on the soybean MAP65-1 events, where plants were exposed to a chilling stress (4° C.) for 10 days, the overall fresh weight of the root systems were protected during chilling challenge, while the controls were significantly affected (FIG. 5).

In summary, 1) soybean MAP65-1 transgenic plants exhibit resistance to P. syringae pv. glycinea LN10; 2) enhanced expression of MAP65-1 elevates resistance to an Oomycete pathogen Phytophthora sojae but not a pathogenic nematode Heterodera glycines; 3) constitutive expression of MAP65-1 transgenic alleles increase the tolerance of soybean to the herbicide oryzalin; and 4) expression of the transgenic MAP65-1 alleles elevate cold and freezing tolerance in soybean.

### Example 17—Sequences

Arabidopsis thaliana MAP65-1
nucleic acid open reading frame
(SEQ ID NO: 1)
ATGGCAGTTACAGATACTGAAAGTCCTCATCTTGGGGAAATTACTTGTG
GTACCTTACTTGAGAAGTTGCAGGAAATCTGGGATGAAGTTGGTGAGAG
TGATGATGAACGAGACAAACTGCTTCTTCAGATAGAGCAAGAGTGTCTT
GACGTTTACAAGAGAAAAGTCGAGCAGGCTGCGAAATCCCGAGCTGAGC
TTCTTCAAACCTTGTCAGATGCTAATGCTGAACTTTCCAGCCTCACAAT
GTCTCTTGGAGACAAAAGCTTAGTTGGCATTCCGGATAAGTCTTCAGGA
ACGATTAAAGAACAACTTGCTGCAATAGCACCGGCTCTTGAACAACTGT
GGCAACAGAAAGAGGAGAGAGTCCGAGAGTTCTCTGATGTACAATCACA
GATTCAGAAGATATGTGGAGATATTGCTGGAGGTTTGAGCAATGAGGTT
CCTATAGTCGATGAGTCTGATTTGTCACTGAAGAAATTAGACGATTTCC
AGAGCCAACTCCAAGAGCTCCAGAAAGAGAAGAGTGACAGGCTGCGCAA
GGTGTTAGAGTTTGTGAGTACTGTTCATGATCTATGTGCTGTTCTTGGT
TTGGATTTCTTAAGCACCGTCACCGAAGTTCATCCGAGCTTAGATGAAG
ATACCAGTGTCCAGTCTAAGAGCATTAGCAATGAGACTCTTTCAAGGTT
GGCTAAAACCGTCTTGACTCTTAAAGATGATAAGAAGCAAAGACTTCAA
AAGCTTCAAGAGCTGGCTACTCAGCTAATTGACCTGTGGAATCTGATGG
ATACTCCTGATGAGGAAAGAGAGCTTTTTGATCATGTTACCTGTAACAT
TTCATCTTCAGTCGATGAGGTCACTGTGCCAGGTGCTCTTGCACGTGAT
TTGATTGAGCAGGTAATTTATATTGCATTGATCAATCTTCCTATGTCCT
CTCTTAGAAACCAGTTACTGCTTGCTAATATTCATGTAAAATTTGTGAA
GGCTGAGGTGGAAGTTGATAGGCTTGACCAGCTGAAAGCTAGCCGAATG
AAAGAAATTGCGTTCAAGAAGCAATCTGAGCTTGAAGAGATATATGCTC
GTGCCCATGTAGAAGTTAACCCGGAATCTGCTCGTGAGAGAATCATGTC
GCTGATTGATTCTGGAAACGTTGAGCCTACTGAATTATTGGCAGACATG
GATAGCCAGATATCAAAGGCTAAGGAAGAAGCATTTAGTAGAAAAGATA
TATTGGACCGAGTCGAGAAATGGATGTCAGCTTGTGAGGAAGAGAGCTG
GCTAGAAGACTACAATCGGGATCAGAACAGGTACAGCGCAAGCAGAGGT
GCACACTTGAATCTCAAGAGAGCTGAGAAAGCTCGGATTCTGGTTAGCA
AGATTCCTGCCATGGTTGACACATTAGTTGCCAAGACCCGGGCTTGGGA
AGAAGAACACAGCATGTCCTTTGCCTACGATGGTGTTCCTCTGCTAGCT
ATGCTAGACGAGTACGGTATGCTTAGGCAAGAACGAGAAGAGGAGAAAC
GGAGGCTGAGGGAACAAAAGAAGGTTCAAGAACAGCCACACGTAGAGCA
AGAATCTGCCTTTAGCACCAGGCCAAGCCCTGCAAGACCGGTCAGTGCT
AAGAAAACGGTGGGGCCACGAGCTAACAACGGAGGAGCCAATGGAACAC
ATAACCGGCGTTTATCTTTGAATGCAAACCAGAATGGAAGCAGGTCTAC
TGCAAAAGAAGCAGGGAGAAGGGAGACTCTCAACAGGCCGGCTGCTCCT
ACAAACTACGTTGCCATTTCGAAAGAGGAAGCTGCTTCATCTCCAGTTT
CTGGTGCTGCAGATCATCAAGTTCCAGCTTCACCA
protein
(SEQ ID NO: 2)
MAVTDTESPHLGEITCGTLLEKLQEIWDEVGESDDERDKLLLQIEQECL
DVYKRKVEQAAKSRAELLQTLSDANAELSSLTMSLGDKSLVGIPDKSSG
TIKEQLAAIAPALEQLWQQKEERVREFSDVQSQIQKICGDIAGGLSNEV
PIVDESDLSLKKLDDFQSQLQELQKEKSDRLRKVLEFVSTVHDLCAVLG
LDFLSTVTEVHPSLDEDTSVQSKSISNETLSRLAKTVLTLKDDKKQRLQ
KLQELATQLIDLWNLMDTPDEERELFDHVTCNISSSVDEVTVPGALARD
LIEQAEVEVDRLDQLKASRMKEIAFKKQSELEEIYARAHVEVNPESARE
RIMSLIDSGNVEPTELLADMDSQISKAKEEAFSRKDILDRVEKWMSACE
EESWLEDYNRDQNRYSASRGAHLNLKRAEKARILVSKIPAMVDTLVAKT
RAWEEEHSMSFAYDGVPLLAMLDEYGMLRQEREEEKRRLREQKKVQEQP
HVEQESAFSTRPSPARPVSAKKTVGPRANNGGANGTHNRRLSLNANQNG
SRSTAKEAGRRETLNRPAAPTNYVAISKEEAASSPVSGAADHQVPASP
Glycine max MAP65-1
nucleic acid open reading frame
(SEQ ID NO: 3)
ATGGCAGTGACCGAAGCTCAAAATCCTCTTCTTGGAGAAAACACATGTG
GTTCCTTGTTAAAAAAGCTTCAGGAAATATGGGATGAGGTTGGTGAGAG
CGATGAGCAACGAGACAAGATGCTTCTTCAGTTAGAACAGGAGTGCTTG
GATGTGTACAAGAGAAAGGTTGAGCAGGCTGCAAAGTCAAGGGCGCAGC
TACTTCAAGCTCTGTCTGATGCTAAGCTTGAGCTTTCCACTCTTCTATC
AGCACTTGGAGAAAAGAGCTTTGCTGGAATTCCTGAGAATACTTCTGGA
ACTATCAAAGAACAGCTTGCAGCTATAGCACCAGTACTTGAACAGTTAT
GGCAACAAAAAGAAGAAAGAATTAAGGAGTTCTCAGATGTACAGTCACA
GATCCAACAAATATGTGGAGAGATAGCCGGGAACTTGAACCTTAATGAT
GTTTCACCTGCAGTTGATGAGTCTGATTTGTCCCTGAAGAAGTTGGATG
AATATCAATCCGAGCTCCAAGAACTTCAAAAGGAAAAGAGCGAGAGGTT
GCACAAGGTTCTTGAATTTGTGAGTACGGTGCATGATCTATGTGCTGTC
CTTGGTATAGACTTCTTCACTACTGTAACTGAGGTTCATCCAAGTCTAA
ATGACTCTACTGGTGTTCAATCCAAGAGCATTAGTAATGACACCCTTGC
AAGGCTGGCTAAGACAGTCTTGACGCTGAAAGAAGATAAAAAACAGAGG
CTGCACAAGCTCCAAGAATTAGCTTCTCAGTTAATTGATCTTTGGAATC
TAATGGATACTCATCCCGAGGAAAGGAGACTATTTGACCATGTTACCTG
TAATATGTCAGCTTCTGTTGATGAAGTCACTGTTCCTGGTGCCCTTGCT
CTGGATCTGATTGAGCAGGCTGAAGTGGAAGTTGAGAGACTTGATCAGC
TGAAAGCCAGCAGGATGAAGGAAATTGCTTTCAAGAAGCAAGCAGAGCT
CGAAGAGATATTTGTCTGTGCTCATATAGAAGTAGATCCAGATGCTGCC
CGGGAGAAGATTATGGCCTTGATTGATTCAGGAAACATTGAACCAACTG
AATTACTGGCTGACATGGACAATCAGATAGCAACAGCAAAAGAAGAAGC
TTTAAGCCGAAAAGATATATTGGACAAGGTTGAGAAATGGATGTCAGCA
TGTGAAGAAGAAAGTTGGCTTGAAGATTATAATCGGGATGATAACAGGT
ATAATGCAAGCAGAGGTGCACACTTAAACCTCAAACGTGCAGAGAAAGC
TCGGATATTGGTCAACAAAATTCCAGCTTTGGTCGATACATTGGTTGCT
AAAACTCGTGCATGGGAAGAAGATCATGGCATGTCATTTACATATGATG
GTGTTCCTCTTCTTGCCATGTTAGATGAATATGCCATGCTCAGACATGA
ACGGGAAGAGGAAAAACGGAGGATGAGGGATCAGAAAAAGCATCACGAG
CAGCGAAACACGGAACAAGAAACCATCTTTGGTTCAAGACCCAGCCCTG
CTAGGCCAGTTAGTTCCAGTAAGTCAGGAGGTCCTCGTGCTAACGGAGG
AGCCAATGGTACTCCTAACCGACGGCTATCGCTTAATGCTCATAAAAAT
GGAAACAGGTCCACATCAAAAGATGGAAAAAGGGATAACAGACTATCTG
CTCCGGTGAATTATGTGGCCATATCAAAAGAAGATGCTGCTTCCCATGT
TTCTGGTACTGAACCCATCCCGGCATCACCC
protein
(SEQ ID NO: 4)
MAVEDTQNPLQGETTCGTLLQKLQEIWDEVGESDEDRDKMLLQIDQECL
DVYKRKVELADKSRAKLLQSLSDAKHELSNLLSALGEKSFSEFPESASG
TIKEQLAAIAPALEQLWKQKEKRVKEFFDVQSEIQKICGEITGSLNISE
NPVVDESQLSVEKLDEYHCQLQELQKEKSERLHKVLEFVSTVHDLCAVL
GIDFFGTVTEVHPSLNDATGVQSKSISNDTLSKLAKMVLALKEDKKQRL
HKLQELATQLVDLWNLMDTPSEERSLFDHVTCNISASVDEVTVPGALAL
DLIEQAEVEVERLDQLKASRMKEIAFKKQSELEEIFAHAHIEIDPEAAR
EKIMTLIDSGNVEPSELLDDMDNKIAKAKEEVLSRKEILDKVEKWMLAC
EEESWLEDYNRDDNRYNASRGAHLNLKRAEKARILVNKIPALVDTLIAK
TRAWEEDRGISFIYDGVPLLAMLDEYTMLRHEREEEKRRFRDHKKFHEQ
QSTDQESITNSRPSPARPIGTKKAAGPRANGAVNGTPSSRRLSLNQNGS
RSMNKDGKRDGMRPMTPVKYDAISKEDSGSHVSGTEPVSSSP

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A construct comprising a nucleic acid sequence encoding a MAP65-1 protein under direction of a promoter.

2. The construct of claim 1, wherein the nucleic acid encoding the MAP65-1 protein comprises the sequence shown in SEQ ID NO:1 or 3 or a sequence having at least 90% sequence identity to SEQ ID NO:1 or 3.

3. The construct of claim 1, wherein the MAP65-1 protein comprises the sequence shown in SEQ ID NO:2 or 4 or a sequence having at least 90% sequence identity to SEQ ID NO: 2 or 4.

4. The construct of claim 1, wherein the promoter is a constitutive promoter.

5. The construct of claim 1, further comprising one or more regulatory elements.

6. A cell comprising the construct of claim 1.

7. The cell of claim 6, wherein the cell is a plant cell.

8. The cell of claim 6, wherein the cell is a bacterial cell.

9. A transgenic plant comprising an exogenous nucleic acid encoding a microtubule-associated protein 65-1 (MAP65-1) protein.

10. The transgenic plant of claim 9, wherein the exogenous nucleic acid encoding the MAP65-1 protein comprises the sequence shown in SEQ ID NO:1 or 3 or a sequence having at least 90% sequence identity to SEQ ID NO:1 or 3.

11. The transgenic plant of claim 9, wherein the transgenic plant exhibits increased resistance to Pseudomonas syringae pv. Glycinea relative to a corresponding plant lacking the exogenous nucleic acid.

12. The transgenic plant of claim 9, wherein the transgenic plant exhibits increased resistance to Phytophthora sojae relative to a corresponding plant lacking the exogenous nucleic acid.

13. The transgenic plant of claim 9, wherein the transgenic plant exhibits increased tolerance to herbicides that destabilize microtubules relative to a corresponding plant lacking the exogenous nucleic acid.

14. The transgenic plant of claim 9, wherein the exogenous nucleic acid further comprises a promoter.

15. The transgenic plant of claim 14, wherein the promoter is a constitutive promoter.

16. The transgenic plant of claim 9, wherein the transgenic plant is a soybean plant.

17. A method of making a transgenic plant, comprising
introducing the construct of claim 9 into a plant cell to generate a transgenic plant cell; and
regenerating the transgenic plant cell into a transgenic plant.

18. The method of claim 17, wherein the transgenic plant exhibits increased resistance to Pseudomonas syringae pv. Glycinea relative to a plant lacking the exogenous nucleic acid encoding the MAP65-1 protein.

19. The method of claim 17, wherein the transgenic plant exhibits increased resistance to Phytophthora sojae relative to a plant lacking the exogenous nucleic acid encoding the MAP65-1 protein.

20. The method of claim 17, wherein the transgenic plant exhibits increased tolerance to herbicides that destabilize microtubules relative to a plant lacking the exogenous nucleic acid encoding the MAP65-1 protein.

21. The method of claim 17, wherein the transgenic plant is a soybean plant.


SEQUENCE LISTING (4 sequences)

SEQ ID NO: 1 - DNA, 1848 bp, Arabidopsis thaliana
  source 1..1848: mol_type = genomic DNA
atggcagttacagatactgaaagtcctcatcttggggaaattacttgtggtaccttacttgagaagttgcaggaaatctgggatgaagttggtgagagtgatgatgaacgagacaaactgcttcttcagatagagcaagagtgtcttgacgtttacaagagaaaagtcgagcaggctgcgaaatcccgagctgagcttcttcaaaccttgtcagatgctaatgctgaactttccagcctcacaatgtctcttggagacaaaagcttagttggcattccggataagtcttcaggaacgattaaagaacaacttgctgcaatagcaccggctcttgaacaactgtggcaacagaaagaggagagagtccgagagttctctgatgtacaatcacagattcagaagatatgtggagatattgctggaggtttgagcaatgaggttcctatagtcgatgagtctgatttgtcactgaagaaattagacgatttccagagccaactccaagagctccagaaagagaagagtgacaggctgcgcaaggtgttagagtttgtgagtactgttcatgatctatgtgctgttcttggtttggatttcttaagcaccgtcaccgaagttcatccgagcttagatgaagataccagtgtccagtctaagagcattagcaatgagactctttcaaggttggctaaaaccgtcttgactcttaaagatgataagaagcaaagacttcaaaagcttcaagagctggctactcagctaattgacctgtggaatctgatggatactcctgatgaggaaagagagctttttgatcatgttacctgtaacatttcatcttcagtcgatgaggtcactgtgccaggtgctcttgcacgtgatttgattgagcaggtaatttatattgcattgatcaatcttcctatgtcctctcttagaaaccagttactgcttgctaatattcatgtaaaatttgtgaaggctgaggtggaagttgataggcttgaccagctgaaagctagccgaatgaaagaaattgcgttcaagaagcaatctgagcttgaagagatatatgctcgtgcccatgtagaagttaacccggaatctgctcgtgagagaatcatgtcgctgattgattctggaaacgttgagcctactgaattattggcagacatggatagccagatatcaaaggctaaggaagaagcatttagtagaaaagatatattggaccgagtcgagaaatggatgtcagcttgtgaggaagagagctggctagaagactacaatcgggatcagaacaggtacagcgcaagcagaggtgcacacttgaatctcaagagagctgagaaagctcggattctggttagcaagattcctgccatggttgacacattagttgccaagacccgggcttgggaagaagaacacagcatgtcctttgcctacgatggtgttcctctgctagctatgctagacgagtacggtatgcttaggcaagaacgagaagaggagaaacggaggctgagggaacaaaagaaggttcaagaacagccacacgtagagcaagaatctgcctttagcaccaggccaagccctgcaagaccggtcagtgctaagaaaacggtggggccacgagctaacaacggaggagccaatggaacacataaccggcgtttatctttgaatgcaaaccagaatggaagcaggtctactgcaaaagaagcagggagaagggagactctcaacaggccggctgctcctacaaactacgttgccatttcgaaagaggaagctgcttcatctccagtttctggtgctgcagatcatcaagttccagcttcacca

SEQ ID NO: 2 - protein, 587 aa, Arabidopsis thaliana
MAVTDTESPHLGEITCGTLLEKLQEIWDEVGESDDERDKLLLQIEQECLDVYKRKVEQAAKSRAELLQTLSDANAELSSLTMSLGDKSLVGIPDKSSGTIKEQLAAIAPALEQLWQQKEERVREFSDVQSQIQKICGDIAGGLSNEVPIVDESDLSLKKLDDFQSQLQELQKEKSDRLRKVLEFVSTVHDLCAVLGLDFLSTVTEVHPSLDEDTSVQSKSISNETLSRLAKTVLTLKDDKKQRLQKLQELATQLIDLWNLMDTPDEERELFDHVTCNISSSVDEVTVPGALARDLIEQAEVEVDRLDQLKASRMKEIAFKKQSELEEIYARAHVEVNPESARERIMSLIDSGNVEPTELLADMDSQISKAKEEAFSRKDILDRVEKWMSACEEESWLEDYNRDQNRYSASRGAHLNLKRAEKARILVSKIPAMVDTLVAKTRAWEEEHSMSFAYDGVPLLAMLDEYGMLRQEREEEKRRLREQKKVQEQPHVEQESAFSTRPSPARPVSAKKTVGPRANNGGANGTHNRRLSLNANQNGSRSTAKEAGRRETLNRPAAPTNYVAISKEEAASSPVSGAADHQVPASP

SEQ ID NO: 3 - DNA, 1746 bp, Glycine max
  source 1..1746: mol_type = genomic DNA
atggcagtgaccgaagctcaaaatcctcttcttggagaaaacacatgtggttccttgttaaaaaagcttcaggaaatatgggatgaggttggtgagagcgatgagcaacgagacaagatgcttcttcagttagaacaggagtgcttggatgtgtacaagagaaaggttgagcaggctgcaaagtcaagggcgcagctacttcaagctctgtctgatgctaagcttgagctttccactcttctatcagcacttggagaaaagagctttgctggaattcctgagaatacttctggaactatcaaagaacagcttgcagctatagcaccagtacttgaacagttatggcaacaaaaagaagaaagaattaaggagttctcagatgtacagtcacagatccaacaaatatgtggagagatagccgggaacttgaaccttaatgatgtttcacctgcagttgatgagtctgatttgtccctgaagaagttggatgaatatcaatccgagctccaagaacttcaaaaggaaaagagcgagaggttgcacaaggttcttgaatttgtgagtacggtgcatgatctatgtgctgtccttggtatagacttcttcactactgtaactgaggttcatccaagtctaaatgactctactggtgttcaatccaagagcattagtaatgacacccttgcaaggctggctaagacagtcttgacgctgaaagaagataaaaaacagaggctgcacaagctccaagaattagcttctcagttaattgatctttggaatctaatggatactcatcccgaggaaaggagactatttgaccatgttacctgtaatatgtcagcttctgttgatgaagtcactgttcctggtgcccttgctctggatctgattgagcaggctgaagtggaagttgagagacttgatcagctgaaagccagcaggatgaaggaaattgctttcaagaagcaagcagagctcgaagagatatttgtctgtgctcatatagaagtagatccagatgctgcccgggagaagattatggccttgattgattcaggaaacattgaaccaactgaattactggctgacatggacaatcagatagcaacagcaaaagaagaagctttaagccgaaaagatatattggacaaggttgagaaatggatgtcagcatgtgaagaagaaagttggcttgaagattataatcgggatgataacaggtataatgcaagcagaggtgcacacttaaacctcaaacgtgcagagaaagctcggatattggtcaacaaaattccagctttggtcgatacattggttgctaaaactcgtgcatgggaagaagatcatggcatgtcatttacatatgatggtgttcctcttcttgccatgttagatgaatatgccatgctcagacatgaacgggaagaggaaaaacggaggatgagggatcagaaaaagcatcacgagcagcgaaacacggaacaagaaaccatctttggttcaagacccagccctgctaggccagttagttccagtaagtcaggaggtcctcgtgctaacggaggagccaatggtactcctaaccgacggctatcgcttaatgctcataaaaatggaaacaggtccacatcaaaagatggaaaaagggataacagactatctgctccggtgaattatgtggccatatcaaaagaagatgctgcttcccatgtttctggtactgaacccatcccggcatcaccc

SEQ ID NO: 4 - protein, 581 aa, Glycine max
MAVEDTQNPLQGETTCGTLLQKLQEIWDEVGESDEDRDKMLLQIDQECLDVYKRKVELADKSRAKLLQSLSDAKHELSNLLSALGEKSFSEFPESASGTIKEQLAAIAPALEQLWKQKEKRVKEFFDVQSEIQKICGEITGSLNISENPVVDESQLSVEKLDEYHCQLQELQKEKSERLHKVLEFVSTVHDLCAVLGIDFFGTVTEVHPSLNDATGVQSKSISNDTLSKLAKMVLALKEDKKQRLHKLQELATQLVDLWNLMDTPSEERSLFDHVTCNISASVDEVTVPGALALDLIEQAEVEVERLDQLKASRMKEIAFKKQSELEEIFAHAHIEIDPEAAREKIMTLIDSGNVEPSELLDDMDNKIAKAKEEVLSRKEILDKVEKWMLACEEESWLEDYNRDDNRYNASRGAHLNLKRAEKARILVNKIPALVDTLIAKTRAWEEDRGISFIYDGVPLLAMLDEYTMLRHEREEEKRRFRDHKKFHEQQSTDQESITNSRPSPARPIGTKKAAGPRANGAVNGTPSSRRLSLNQNGSRSMNKDGKRDGMRPMTPVKYDAISKEDSGSHVSGTEPVSSSP